(19) United States
(12) Patent Application Publication
(10) Pub. No.: US 2023/0134136 A1
(43) Pub. Date: May 4, 2023

(54) METHODS OF TREATING VIRAL INFECTIONS AND HEALTH CONSEQUENCES

(71) Applicant: Allen DAVIDOFF, Calgary (CA)

(72) Inventor: Allen Davidoff, Calgary (CA)

(21) Appl. No.: 17/911,789

(22) PCT Filed: Mar. 15, 2021
(86) PCT No.: PCT/IB2021/000238

§ 371 Date: Sep. 15, 2022

Related U.S. Application Data
(60) Provisional application No. 62/989,879, filed on Mar. 15, 2020.

(51) Int. Cl. A61K 38/44; A61K 45/06; A61P 13/12; A61P 31/14; C12N 9/06

(52) U.S. Cl. CPC A61K 38/44; A61K 45/06; A61P 13/12; A61P 31/14; C12N 9/0048; C12Y 107/03003

(57) ABSTRACT

This invention relates to formulations of uric acid lowering agent(s) (UALA) designed to inhibit xanthine oxidase and/or decrease serum or tissue uric acid concentration for the treatment and prevention of morbidities and mortality during viral infection. For example, acute kidney injury due to coronavirus infection by administering a therapeutically effective amount of an agent capable of inhibiting xanthine oxidase and/or decreasing uric acid levels in a patient in need of such treatment. Additionally, the scope of the invention includes a method of treating and preventing acute kidney injury and health consequences due to coronavirus infection.

### FIELD

The invention relates to compositions, methods, and uses for uric acid lowering agents in the setting of viral infection, and health consequences of that viral infection. The invention also relates to methods for decreasing aberrant purine metabolism, decreasing circulating concentration of uric acid, decreasing enzymatic production of uric acid including inhibition of xanthine oxidase activity, and compositions comprising the agents and uses of such agents, and methods for the treatment of diseases and acute conditions using the salts, formulations and compositions.

### BACKGROUND

Coronavirus infections such as SARS, MERS and Covid-19 represent a new vector of infection that can lead to severe pulmonary, vascular, and renal injury. Similarly, viral infections such as SARS, MERS and Covid-19 represent a new vector of infection that can lead to severe inflammatory responses of a subject and involve organ systems such as pulmonary, vascular, cardiovascular, central nervous system, pancreas, and renal. In addition, some studies suggest that virus infection can lead to a number of secondary physiologic challenges due to increased activation of the inflammatory response, a pro-coagulative environment, immune responsiveness, and susceptibly to secondary bacterial pneumonia.

### BRIEF DESCRIPTION OF THE DRAWINGS

FIG. 1 is a graph showing that patients with COVID-19 show signs of acute kidney injury (AKI) and accompanying hyperuricemia. A dose-dependent correlation between serum uric acid concentration and acute kidney injury in patients infected with COVID-19 Coronavirus is observed.

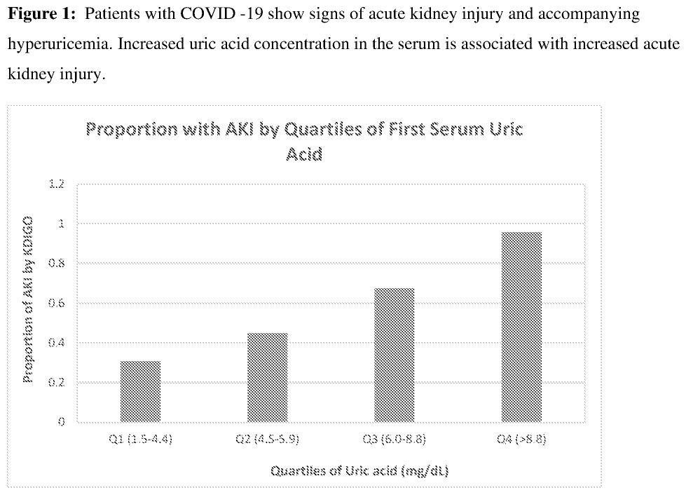

FIG. 2 is a graph showing that patients infected with COVID-19 Coronavirus with normal kidney function compared to acute kidney injury—using MAKE Criteria—show a distinction regarding concentrations of serum uric acid. MAKE Criteria is defined as a 2-fold increase in creatinine concentration in serum, the need for dialysis or death. Hyperuricemia is present in individuals with AKI.

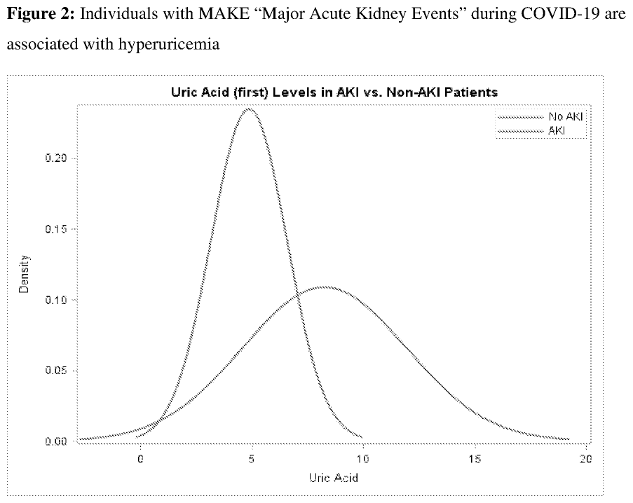

FIG. 3 shows that during COVID-19 infection hyperuricemia is associated with an increased Hazard Ratio for acute kidney injury. Approximately 60% of hospitalized COVID-19 infected individuals manifest hyperuricemia compared to approximately 20% in the health population. Increased uric acid concentration is dose dependently associated with an increased in Hazard Ratio.

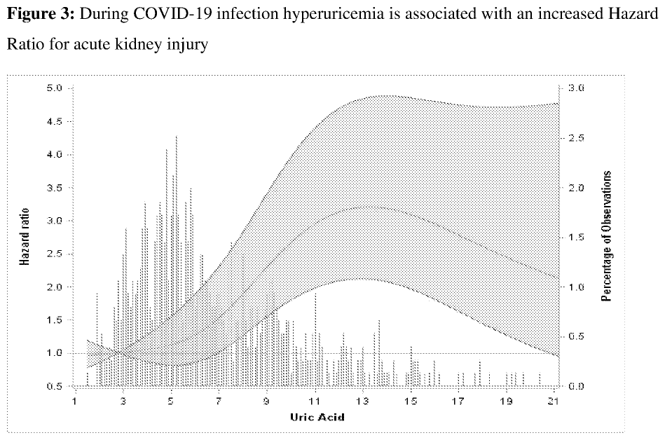

FIG. 4 in individuals hospitalized with COVID-19 infection, hyperuricemia is associated with increased troponin release—a marker of cardiac injury. Patients infected with COVID-19 show increased Troponin concentration in blood samples that is dose dependently associated with uric acid concentration.

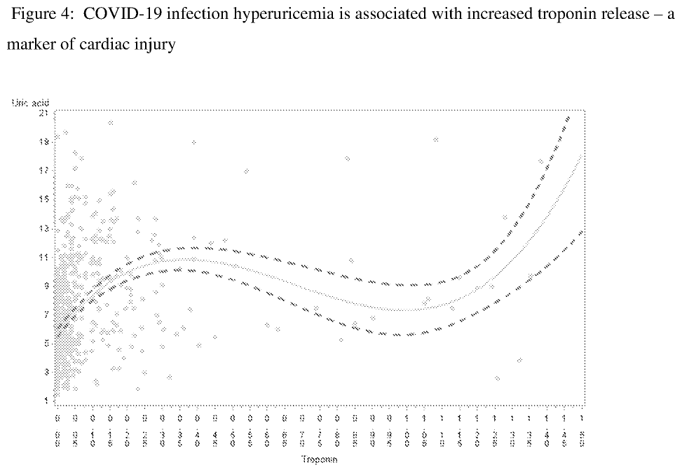

FIG. 5 in hospitalized individuals with confirmed COVID-19 infection, hyperuricemia is associated with increased circulation concentrations of procalcitonin—an indicator of inflammatory state and of cellular lysis cytosolic metabolic products and/or cellular debris.

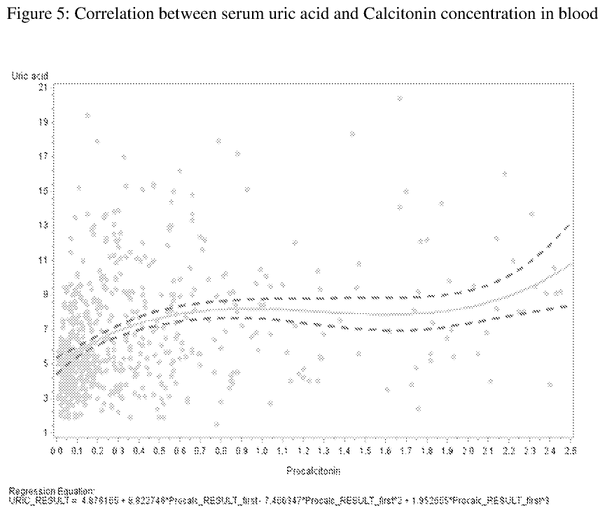

FIG. 6 patients with confirmed COVID-19 infection were treated with a uric acid lowering agent—rasburicase—show decreased severity of acute kidney injury.

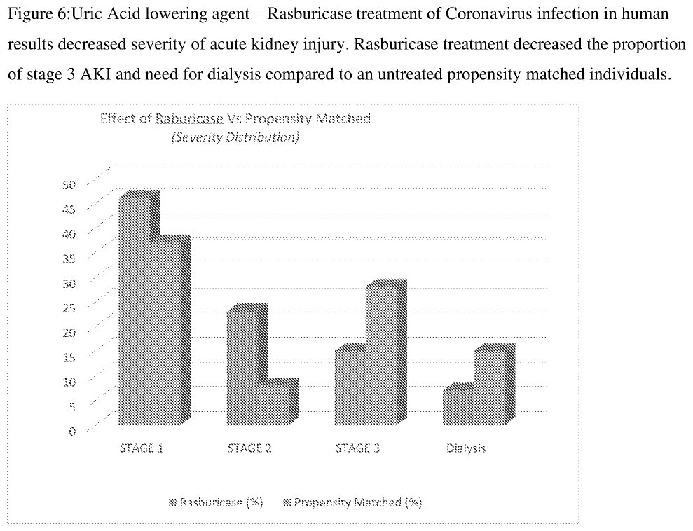

FIG. 7 is a diagram of adenosine catabolism and generation of oxygen-free radicals in influenza virus-infected lung. XO, which is the final enzyme in purine catabolism, transfers electrons to molecular oxygen to form superoxide anion (O2). 0″ can be converted into highly toxic hydroxyl radicals by the iron-catalyzed Haber-Weiss reaction (16). Square boxes indicate purine metabolites. Enzymes involved are shown in rounded boxes. Allopurinol inhibits XO.

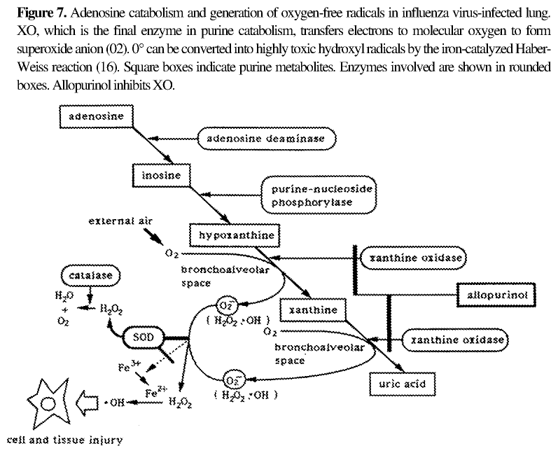

### DETAILED DESCRIPTION

The novel invention is the use of a uric acid lowering agent (UALA), or agents, alone, or in combination with basic organic molecules to ameliorate symptoms of pulmonary, vascular and/or nephrology associated with or caused by viral, or coronavirus, or COVID-19 infection.

In a second aspect of the disclosure, a uric acid lowering agent to decrease the production, reuptake or increase breakdown as a means of decreasing circulating uric acid and uric acid crystal formation.

In a third aspect of the disclosure, a basic organic or inorganic molecule to increase serum or urine pH and therefore decrease uric acid solubility, decreasing the ability of uric acid crystals to form.

In a fourth aspect of the disclosure, oxypurinol will have anti-viral activity diminishing the potency of coronavirus infection, morbidity and mortality.

In a fifth aspect of the disclosure, the combination of a uric acid lowering agent(s) and an antiviral drug, a synthetic nucleoside analogue, that has inhibitory activity (interferes with viral replication). Nucleoside analogs represent the largest class of small molecule-based antivirals, which currently form the backbone of chemotherapy of chronic infections caused by HIV, hepatitis B or C viruses, and herpes viruses. High antiviral potency and favorable pharmacokinetics parameters make some nucleoside analogs suitable also for the treatment of acute infections caused by other medically important RNA and DNA viruses. For example: acyclovir, remdesivir,

It has been discovered that elevated levels of uric acid is a primary mediator of acute kidney injury, and/or acute cardiac injury and/or other morbidities associated with COVID-19 infection. The disclosure provides a new approach to combating the epidemic of viral infection and resulting co-morbidities and mortality. In one embodiment, the disclosure provides an approach to preventing and/or treating one or more coronavirus related characteristics.

In a specific embodiment, the subject disclosure pertains to methods of administering a uric acid lowering agent (UALA), or agents, (e.g. uricase & xanthine oxidase inhibitor together or sequential administration of uricase then xanthine oxidase inhibitor) to a patient susceptible to developing COVID associated characteristics. As part of the medical treatment, serum samples may be obtained and tested so the serum uric acid levels may be monitored in conjunction with the administration of the UALA.

In another embodiment, provided is an approach to preventing and/or treating COVID-19 related acute kidney injury. In a specific embodiment, the subject of the disclosure pertains to methods of administering uric acid lowering agent (UALA) to a patient susceptible to developing or suffering from COVID-19 infection.

In another embodiment, the subject disclosure provides an approach to reducing the risk of developing, delaying the onset of and/or treating acute cardiac injury or chronic cardiac injury.

In another embodiment, provided is an approach to reducing the risk of endothelial and or vascular injury and fibrosis or calcification of vascular tissue.

In another embodiment, provided is an approach to reducing the effects of xanthine oxidase and/or increased uric acid in the setting of coronavirus and COVID-19 or other viral infections.

In another embodiment, the provided is an approach to decrease the hyperuricemia defined as uric acid greater than 5.5 mg/dL, in acute or chronic pancreatic injury and/or viral diabetes and the health consequences of viral diabetes, and/or in the setting of coronavirus and or COVID-19 infection.

In another embodiment, the subject of the disclosure provides an approach to decrease the hyperuricemia defined as uric acid greater than 5.5 mg/dL, in acute or chronic hepatic injury in the setting of coronavirus and or COVID-19 infection.

In another embodiment, provided is use of uric acid lowering agents such as uricase based therapeutics to decrease serum uric acid, to treat and prevent acute organ injury and or acute injury to a variety of body systems in the setting of viral, coronavirus and/or COVID-19 infection.

In another embodiment, the disclosure provides uric acid lowering agents such as xanthine oxidase inhibitor based therapeutics to decrease serum uric acid, to treat and prevent acute and/or chronic organ injury and or acute and/or chronic injury to a variety of body systems in the setting of coronavirus and or COVID-19 infection.

In another embodiment, the subject disclosure provides uric acid lowering agents such as uricosuric agent based therapeutic to decrease serum uric acid, to treat and prevent acute and/or chronic organ injury and or acute and/or chronic injury to a variety of body systems in the setting of coronavirus and or COVID-19 infection.

In another embodiment, the subject disclosure provides uric acid lowering agents in a combination of uricase, xanthine oxidase inhibitor or uricosuric based therapeutic—administered simultaneously or consecutively to decrease serum uric acid, to treat and prevent acute and/or chronic organ injury and or acute and/or chronic injury to a variety of body systems in the setting of coronavirus and or COVID-19 infection.

In another embodiment, the subject disclosure provides uric acid lowering agents in a combination of an amino acid within the uric acid pathway— L-Arginine, L-citrulline and or L-ornithine, and/or a basic amino acid, uricase, xanthine oxidase inhibitor or uricosuric based therapeutic—administered simultaneously or consecutively to decrease serum uric acid, to treat and prevent acute and/or chronic organ injury and or acute and/or chronic injury to a variety of body systems in the setting of coronavirus and or COVID-19 infection.

In another embodiment, the subject disclosure provides a method for treating or preventing acute respiratory distress syndrome with the use of a uric acid lowering agent.

In another embodiment, the subject disclosure provides a method of treating acute cardiac injury due to hyperuricemia using a uric acid lowering agent in the setting of viral infection and/or sepsis and/or acute respiratory distress syndrome.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present disclosure are generally performed according to conventional methods well known in the art and as described in various general and more specific references that are cited and discussed through the present specification unless otherwise indicated.

It is to be understood that the recitation of numerical ranges by endpoints includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” Further, it is to be understood that the singular forms “a”, “an”, and “the” include plural references unless the context clearly dictates otherwise. Thus, for example, reference to “an organic base” includes one or more organic bases and equivalents thereof known to those skilled in the art, and so forth.

Some of the compounds described herein contain one or more asymmetric centers and may give rise to enantiomers, diasteriomers, and other stereoisomeric forms which may be defined in terms of absolute stereochemistry as (R)- or (S)-. The present disclosure is meant to include all such possible diasteriomers and enantiomers as well as their racemic and optically pure forms. Optically active (R)- and (S)-isomers may be prepared using chiral synthons or chiral reagents, or resolved using conventional techniques such as chiral HPLC. When the compounds described herein contain centers of geometric asymmetry, and unless specified otherwise, it is intended that the compounds include both E and A geometric isomers. All tautomeric forms are intended to be included within the scope of the disclosure.

Particular stereoisomeric forms described in this disclosure are meant to be substantially free of any other stereoisomeric configuration. Substantially free means that the active ingredient contains at least 80%, 85%, 90%, and 95% by weight of the desired stereoisomer and 20%, 15%, 10%, and 5% by weight or less of other stereoisomers, respectively. In particular, the weight % ratio is greater than 95:5 and most preferably 99:1 or greater.

The term “about” means plus or minus 1%, 2%, 3%, 4%, 5%, 6%, 7%, 8%, 9%, or 10% of the number to which reference is being made.

The terms “administering” or “administration” of an agent, drug, or peptide to a subject refers to any route of introducing or delivering to a subject a compound to perform its intended function. The administering or administration can be carried out by any suitable route, including orally, intranasally, parenterally (intravenously, intramuscularly, intraperitoneally, or subcutaneously), rectally, or topically. Administering or administration includes self-administration and the administration by another.

The terms “co-administration”, “co-administered” or “co-administering” as used herein refer to the administration of a substance before, concurrently, or after the administration of another substance such that the biological effects of either substance overlap.

The term “amino acid” refers to naturally occurring and synthetic α, β γ or δ amino acids, and includes but is not limited to, amino acids found in proteins, i.e. glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine. In certain embodiments, the amino acid is in the L-configuration. Alternatively, the amino acid can be a derivative of alanyl, valinyl, leucinyl, isoleuccinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, isoleuccinyl, 3-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl.

“Basic amino acids” include arginine, lysine, and ornithine. “Arginine” refers to the naturally occurring L-amino acid, any biochemical equivalents, and any precursors, basic forms, functionally equivalent analogs, and physiologically functional derivatives thereof. It includes sulfates of L-arginine, and sulfates of its functional analogs. Derivatives include peptides (i.e. poly L-arginine, arginine oligomers), other nitric oxide precursors such as homoarginine or substituted arginine such as hydroxyl-arginine. Therefore, suitable arginine compounds that may be used in the present disclosure include but are not limited to L-arginine, D-arginine, DL-arginine, L-homoarginine, and N-hydroxy-L-arginine, including their nitrosated and nitrosylated analogs (for example, nitrosated L-arginine, nitrosylated L-arginine, nitrosated N-hydroxy L-arginine, nitrosylated N-hydroxy-L-arginine, nitrosated L-homoarginine, and nitrosylated L-homoarginine, precursors of L-arginine and/or physiologically acceptable salts thereof, including for example, citrulline, ornithine, glutamine, lysine, polypeptides comprising at least one of these amino acids, and inhibitors of the enzyme arginase (e.g. N-hydroxy-L-arginine, and 2(S)-amino boronohexalioic acid). Naturally occurring sources include protamine. An arginine compound may be selected that lowers serum lipid.

“Lysine” refers to a naturally occurring L-amino acid any biochemical equivalents, and any precursors, basic forms, functionally equivalent analogs, and physiologically functional derivatives thereof. It includes sulfates of L-lysine, and sulfates of its functional analogs. Derivatives include peptides (i.e. poly L-lysine, lysine oligomers), other such as homolysine, L-N⁶-(1-iminoethyl)lysine derivatives, or substituted lysine such as methylated lysine, hydroxylysine, lysine substituted with an N-epsilon-alkoxy or N-epsilon-alkenoxycarbonyl group, lysine substituted with a N^{c}-fluoroalkyloxycarbonyl or N^{c}-fluoroalkylsulphonyl group, lysine substituted with N^{x}-(2-Nitropenylthio)-N-epsilon-acyl, or lysine substituted with a N-alkylsulphonyl or alkyl-aminocarbonyl group. Therefore, suitable lysine compounds that may be used in the present disclosure include but are not limited to L-lysine, D-lysine, DL-lysine, 6,6-dimethyl lysine, L-homolysine, and N-hydroxy-L-lysine, N-epsilon-2-hexyldecyloxycarbonyl-L-lysine, N-epsilon-2-decyltetradecyloxycarbonyl-L-lysine, N-epsilon-tetradecyloxycarbonyl-L-lysine, N-epsilon-2-hexadecyloxy-N-epsilon-2-hexyldecyloxycarbonyl-L-lysine, L-N⁶-(1-iminoethyl)lysine, N-epsilon-2-decyltetradecyloxycarbonyl-L-lysine, N-epsilon-tetradecyloxy-carbonyl-L-lysine, N^{c}-2-(F-octyl)ethyloxycarbonyl-L-lysine or N^{c}-2-(F-hexyl)ethyloxycarbonyl-L-lysine, N-epsilon-dodecylsulphonyl-L-lysine, N-epsilon-dodecylamino-carbonyl-L-lysine, including their nitrosated and nitrosylated analogs (for example, nitrosated L-lysine, nitrosylated L-lysine, nitrosated N-hydroxy L-lysine, nitrosylated N-hydroxy-L-lysine, nitrosated L-homo lysine, and nitrosylated L-homolysine, precursors of L-lysine and/or physiologically acceptable salts thereof. Lysine, and analogs and derivatives thereof may be prepared using methods known in the art or they may be obtained from commercial sources. For example, L-lysine is commercially produced utilizing gram positive Corynebacterium glutamicum, Brevibacterium flavum and Brevibacterium lactofermentum (Kleemann, A., et. al., “Amino Acids,” in ULLMANN'S ENCYCLOPEDIA OF INDUSTRIAL CHEMISTRY, vol. A2, pp. 57-9′7, Weinham: VCH-Verlagsgesellschaft (1985)), or mutant organisms.

The term “organic base” refers to a hydrocarbon base. An organic base that enhances the solubility of a particular UALA may be selected for use in a composition of the invention. A pharmaceutically acceptable organic base is generally selected for use in the present disclosure. The organic base can be a solubilizing compound that increases the aqueous solubility of a target UALA. A solubilizing compound may be a hydrotropic agent that increases the affinity of a target UALA for water. The concentration and/or solubility of a UALA in a composition of the disclosure can be greater in the presence of the hydrotropic agent than in its absence. A hydrotropic agent may be characterized by one or more of the following:
a) comprises at least one hydrophobic moiety;
b) high water solubility (e.g. at least 2M);
c) destabilizes water structure and at the same time interacts with a poorly soluble drug;
d) at high concentrations solubilize a poorly soluble drug in water;
e) self-associates and forms noncovalent planar or open-layer structures;
f) nonreactive;
g) non-toxic; and/or
h) does not produce any temperature effect when dissolved in water;

An organic base may be a Class 1, Class 2, or Class 3 organic base as described in the “Handbook of Pharmaceutical Salts, Properties, Selection and Use” P. Heinrich Stahl and Camille G Wermuth (Eds), Published by VHCA (Switzerland) and Wiley-VCH (FRG), 2011.

In specific embodiments, the organic base may be (a) a Class 1 base with a pKa1 between about 7 to 13 including but not limited to L-arginine, D-arginine, choline, L-lysine, D-lysine, and caffeine.

The term “coronavirus” refers to Coronaviruses (CoVs) that constitute a group of phylogenetically diverse enveloped viruses that encode the largest plus strand RNA genomes and replicate efficiently in most mammals. Human CoV (HCoVs-229E, OC43, NL63, and HKU1) infections typically result in mild to severe upper and lower respiratory tract disease. Severe Acute Respiratory Syndrome Coronavirus (SARS-CoV) emerged in 2002-2003 causing acute respiratory distress syndrome (ARDS) with 10% mortality overall and up to 50% mortality in aged individuals. Middle Eastern Respiratory Syndrome Coronavirus (MERS-CoV) emerged in the Middle East in April of 2012, manifesting as severe pneumonia, acute respiratory distress syndrome (ARDS) and acute renal failure. Severe Acute Respiratory Syndrome Coronavirus 2 (SARS-CoV-2) emerged in 2019 causing coronavirus disease 2019 (COVID-19) and the COVID-19 pandemic.

A “condition” and/or “disease” contemplated herein refers to a condition and/or disease which requires modulation of uric acid lowering agent or xanthine oxidase or which utilizes xanthine oxidase inhibitors to treat or prevent the condition or disease. In particular applications the condition or disease is a acute or chronic cardiovascular disease and related diseases, ischaemia-reperfusion injury in tissues including the heart, lung, kidney, gastrointestinal tract, and brain, diabetes, inflammatory joint diseases such as rheumatoid arthritis, respiratory distress, kidney disease, liver disease, sickle cell disease, sepsis, burns, viral infections, hemorrhagic shock, gout, hyperuricaemia, and conditions associated with excessive resorption of bone.

Cardiovascular and related diseases include, for example, hypertension, hypertrophy, congestive heart failure, heart failure subsequent to myocardial infarction, arrhythmia, myocardial ischemia, myocardial infarction, conditions associated with poor cardiac contractility, conditions associated with poor cardiac efficiency, ischemia reperfusion injury, and diseases that arise from thrombotic and prothrombotic states in which the coagulation cascade is activated.

The term “dose” refers to a measured quantity of a medicine, nutrient, or pathogen which is delivered as a unit. A “unit dosage” refers to a unitary i.e. single dose, which comprises all the components of a composition of the disclosure, which is capable of being administered to a patient. A “unit dosage” may be readily handled and packed, remaining as a physically and chemically stable unit dose comprising the active agent and/or organic base with pharmaceutical carriers, excipients, vehicles, or diluents.

The term “Therapeutically effective amount” relates to a dose of the substance that will lead to the desired pharmacological and/or therapeutic effect. The desired pharmacological effect is, to alleviate a condition or disease described herein, or symptoms associated therewith. A therapeutically effective amount of a substance may vary according to factors such as the disease state, age, sex, and weight of the individual, and the ability of the substance to elicit a desired response in the individual. Dosing regimen may be adjusted to provide the optimum therapeutic response. For example, several divided doses may be administered daily, or the dose may be proportionally reduced as indicated by the exigencies of the therapeutic situation.

The term “health consequences” refers to but is not limited to fevers, coughs, myalgia or fatigues, and atypical symptoms included sputum, headache, hemoptysis (coughing up blood), diarrhea, and other morbidities associated with coronavirus or COVID-19 infection. Severe health consequences include acute respiratory distress syndrome, acute heart injury, acute renal injury, acute neurologic injury, acute pancreatic injury, and acute liver injury or chronic injuries that follow COVID-19 infection.

“Acute Kidney Injury (AKI)” refers to any impairment of kidney function as described by “MAKE” criteria, “KIDGO” criteria, uric output, increase in creatinine concentration in serum, increase in proteinuria in urine, decrease in glomerular filtration rate and can be calculated by eGFR or other calculation that result in a similar measure, increase in local inflammation in the kidney, any stage of acute kidney injury—for example stage 1, 2, or 3, the need for dialysis or as a cause of mortality or other health consequences.

“Acute Cardiac Injury” refers to any impairment of cardiac function that decreases the energetic efficiency of the heart and is characterized by a balance between left ventricular performance and myocardial energy consumption. Cardiac efficiency may be assessed by the ratio of stroke work (SW) to myocardial oxygen consumption per unit time (MVO2). Stroke work (SW) can be calculated as the area of the pressure-volume loop of the cardiac cycle. Myocardial oxygen consumption (MVO2) can be calculated from myocardial oxygen extraction (AVO2), left main coronary blood flow (Qcor), and blood hemoglobin concentration with the Fick equation. Left main coronary blood flow (Qcor) can be calculated from the coronary flow velocity and left main diameter assuming laminar flow (Doucette, J W et al, Circulation 1992; 85:1899-1911). Acute Cardiac Injury includes increases in measures of troponin measured in the serum and used an indicator of injury to cardiomyocytes.

In an aspect, MVO2 may be determined using the Fick's equation using a coronary sinus venous blood sample, arterial blood sample, and coronary blood flow. Coronary blood flow may be measured with a thermodilution technique. In this case, MVO2=(CaO2−CvO2)×CBF where CaO2 is the arterial oxygen content, CvO2 is the coronary sinus oxygen content and CBF is the coronary blood flow. Myocardial oxygen extraction (AVO2) is calculated as the difference between arterial and coronary sinus O2 saturations.

Improved cardiac efficiency refers to a decrease in oxygen consumption (MVO2) associated with an increase in mechanical efficiency or efficiency of myocardial contraction. Efficiency of myocardial contraction can be assessed by determining the peak rate of rise of left ventricular pressure (dP/dTmax). A decrease in oxygen consumption may represent a 1-70% decrease, in particular a 1%, 2%, 3%, 4%, 5%, 6%, 7%, 8%, 9%, 10%, 15%, 20%, 25%, 30%, 35%, 40%, 50%, 60%, or 70% decrease in oxygen consumption. An increase in mechanical efficiency or efficiency of contraction may represent a 1-70% increase, in particular a 1%, 2%, 3%, 4%, 5%, 6%, 7%, 8%, 9%, 10%, 15%, 20%, 25%, 30%, 35%, 40%, 50%, 60%, or 70% increase in mechanical efficiency or efficiency of contraction. The decrease in oxygen consumption and/or increase in mechanical efficiency may be significant.

In an aspect, improved cardiac efficiency is represented by an increase in SW/MVO2. An increase in SW/MVO2 may represent a 1-70% increase, in particular a 1%, 2%, 3%, 4%, 5%, 6%, 7%, 8%, 9%, 10%, 15%, 20%, 25%, 30%, 35%, 40%, 50%, 60%, 70%, 30-70%, or 40-60% increase in SW/MVO2.

In particular embodiments, the decrease in oxygen consumption or increase in mechanical efficiency or contraction, or increase in SW/MVO2 or cardiac efficiency is significant or statistically significant. The term “significant” or “statistically significant” refers to statistical significance and generally means a two standard deviation (SD) above or below standard or normal, or a higher or lower concentration of the element.

“Acute Vascular Injury” Includes any injury to endothelial cells lining a blood vessel, or injury to smooth muscle cells attributable directly to viral infection and more specifically to lytic viral infection, coronavirus infection or COVID-19 infection or viral infections due to strains of those viruses. Acute vascular injury can also refer to indirect effects of vascular injury including increase in vascular tone, vasoconstriction, vasodilation, high blood pressure, unstable blood pressure, lysis of endothelial cells, decrease in endothelial progenitor cells, pro-inflammatory or pro-coagulative measures and indices of clotting associated with viral infection and hyperuricemia.

“Acute Neurological Injury”—includes any injury to brain, blood brain barrier, nervous system,neurological vascular supply, inflammation, stroke, dementia, hallucinations, neurological lymphatic system, neurological limbic system, chronic fatigue or, neuropathy or neuropathic pain associated with viral infection, inflammation, thrombotic or inflammatory injury directly or indirectly associated with viral injury.

The terms “subject”, “individual” or “patient” refer to an animal including a warm-blooded animal such as a mammal, which is afflicted with or suspected of having or being pre-disposed to a condition or disease as described herein. In particular, the terms refer to a human. The terms also include domestic animals bred for food or as pets, including horses, cows, sheep, poultry, fish, pigs, cats, dogs, and zoo animals.

The methods herein for use on subjects/individuals/patients contemplate prophylactic and therapeutic or curative use. Typical subjects for treatment include persons susceptible to, suffering from or that have suffered a condition or disease described herein. In particular, suitable subjects for treatment in accordance with the invention include persons that are susceptible to, suffering from or that have obesity, insulin resistance, metabolic syndrome, pre-diabetes, diabetes, kidney disease, heart disease, heart failure, or acute cardiogenic shock. In particular aspects of the invention patients are selected where a uric acid lowering agent will decrease serum uric acid concentration and the health consequences of such is desirable.

The terms “preventing or treating” and “prophylactic and therapeutic” refer to administration to a subject of biologically active agents either before or after onset of a condition or disease. If the agent is administered prior to exposure to a factor causing a condition or disease the treatment is prophylactic (i.e. protects the host against damage). If the agent is administered after exposure to the factor causing a condition or disease the treatment is therapeutic (i.e. alleviates the existing damage). A treatment may be either performed in an acute or chronic way.

The terms “pharmaceutically acceptable carrier, excipient, vehicle, or diluent” refer to a medium which does not interfere with the effectiveness or activity of an active ingredient and which is not toxic to the hosts to which it is administered. A carrier, excipient, vehicle, or diluent includes but is not limited to binders, adhesives, lubricants, disintegrates, bulking agents, buffers, and miscellaneous materials such as absorbents that may be needed in order to prepare a particular composition.

The terms “uricase” refers to Urate oxidase (Uricase EC 1.7.3.3, uox) which is a homotetrameric enzyme composed of four identical 34 KDa subunits. The enzyme is responsible for the initial step that begins a series of reactions that convert uric acid to a more soluble and easily excreted product, allantoin. In short, uricase catalyzes the reaction of uric acid (UA) with O₂ and H₂O to form 5-hydroxy-isourate (HIU) and the release of H₂O₂. HIU is an unstable product that undergoes non-enzymatic hydrolysis to 2-oxo-4-hydroxy-4-carboxy-5-ureidoimidazoline (OHCU) and then decarboxylates spontaneously to form racemic allantoin. In species that contains a functional uricase, two additional enzymes are expressed (HIU hydrolase and OHCU decarboxylase) which catalyze these reaction more quickly to generate (s)-allantoin. A functional uricase can be found in a wide range of organisms: archaea, bacteria, and eukaryotes. However, in humans and some primates a functional uricase is not expressed. The lack of uricase expression is attributed to three genetic mutations: a nonsense mutation at codon 33 (impacting orangutans, gorillas, chimpanzees, and humans), another nonsense mutation at codon 187 (impacting chimpanzees and humans) and a mutation at the splice acceptor site in intron 2 (impacting chimpanzees and humans). Lastly, it has been shown that uricase treatment rapidly reduces UA levels in the peripheral blood stream by oxidizing UA to a more soluble product, allantoin.

The terms “uricosuric agents” or “uricosuric based therapeutics” refer to molecules that increase the excretion of uric acid in the urine, thus reducing the concentration of uric acid in blood plasma. The uricosuric agents act on the proximal tubules in the kidneys, where they interfere with the absorption of UA from the kidney back into the blood. Uricosuric based theraputics, such as Benzbromarone and Lesinurad, promote excretion of UA.

The term “viral infection” refers to any stage of viral life cycle and to viral infection and more specifically to lytic viral infection, coronavirus infection or COVID-19 infection or viral infections due to strains of those viruses that is associated with hyperuricemia, either transient, intermittent or permanent due to soluble or crystalluria effects.

The term “Xanthine oxidase inhibitor” refers to compounds that inhibit xanthine oxidase. Methods known in the art can be used to determine the ability of a compound to inhibit xanthine oxidase. (See for example the assay described in U.S. Pat. No. 6,191,136). A number of classes of compounds have been shown to be capable of inhibiting xanthine oxidase, and medicinal chemists are well aware of those compounds and manners in which they may be used for such purpose. It will be appreciated by the skilled artisan that xanthine oxidase inhibitors are numerous, and that the present disclosure may be carried out with any of the classes of pharmaceutically acceptable xanthine oxidase inhibitors.

Functional derivatives of a xanthine oxidase inhibitor can be used in certain embodiments. “Functional derivative” refers to a compound that possesses a biological activity (either functional or structural) that is substantially similar to the biological activity of a xanthine oxidase inhibitor. The term “functional derivative” is intended to include “variants” “analogs” or “chemical derivatives” of a xanthine oxidase inhibitor. The term “variant” is meant to refer to a molecule substantially similar in structure and function to a xanthine oxidase inhibitor or a part thereof. A molecule is “substantially similar” to a xanthine oxidase inhibitor if both molecules have substantially similar structures or if both molecules possess similar biological activity. The term “analog” refers to a molecule substantially similar in function to a xanthine oxidase inhibitor. The term “chemical derivative” describes a molecule that contains additional chemical moieties which are not normally a part of the base molecule. A derivative may be a “physiological functional derivative” which includes but is not limited to a bioprecursor or “prodrug” which may be converted to a xanthine oxidase inhibitor.

A representative class of xanthine oxidase inhibitors for use in the compositions of the present invention are disclosed in U.S. Pat. Nos. 6,191,136 and 6,569,862, which are incorporated herein by reference. Compounds that are particularly useful include allopurinol (4-hydroxy-pyrazolo[3,4-d]pyrimidine) or oxypurinol (4,6-dihydroxypyrazolo[3,4-d]pyrimidine], or tautomeric forms thereof. Xanthine oxidase inhibitors for use in the present disclosure can be synthesized by known procedures. Some therapeutic xanthine oxidase inhibitors also are commercially available, such as allopurinol, febuxostat and oxypurinol. A xanthine oxidase inhibitor may be in a non-crystalline form, or a crystalline or amorphous form, or it may be a pharmaceutically acceptable salt of a xanthine oxidase inhibitor.

### Overview

While there are no current examples of pulmonary or renal xanthine oxidase expression in the case of coronavirus infections, tissue injury, infection and tissue lysis can lead to increased circulating nucleic acid (cell free DNA) and uric acid concentrations. Increased circulating nucleic acid concentrations are quickly converted into uric acid, which in turn can trigger sudden and overwhelming accumulation of uric acid crystals and thereafter cause acute injury to varied body systems and importantly acute kidney injury.

Hyperuricemia is reported to contribute to acute organ and more specifically kidney injury in the setting of cardiac surgery, crushing trauma to tissues and in the setting of tumor lysis syndrome. When viral, coronavirus or COVID-19 infection is present, tissue lysis and subsequent hyperuricemia is not known to contribute to acute organ or acute kidney injury.

Inhibitors of the enzyme xanthine oxidase, which converts hypoxanthine to xanthine, and xanthine to uric acid, have been indicated for the treatment of a variety of conditions. For example, the xanthine oxidase inhibitor, allopurinol, is used in the treatment of gout and hyperuricaemia (U.S. Pat. No. 5,484,605). Xanthine oxidase inhibitors have also been proposed for use in suppressing the harmful effects of oxygen radicals that mediate ischaemia-reperfusion injury in a variety of tissues including the heart, lung, kidney, gastrointestinal tract, and brain, and in inflammatory joint diseases such as rheumatoid arthritis. (See for example, U.S. Pat. No. 6,004,966). They have also been reported to be useful in treating excessive resorption of bone. (U.S. Pat. No. 5,674,887). Further, allopurinol, oxypurinol, and other xanthine oxidase inhibitors have been found to be effective in the treatment of congestive heart failure (U.S. Pat. No. 6,569,862).

In the setting of viral infection that leads to acute injury, uric acid lowering agents can decrease hyperuricemia and the health consequences of hyperuricemia. Increased inflammatory, coagulative state, oxidative state, hypercatabolic state, rhabdomyolysis or acute respiratory syndrome may be addressed directly by uric acid lowering agents or by combinations of uric acid lowering agents or anti-oxidants, or anti-inflammatory agents.

The citation of any reference herein is not an admission that such reference is available as prior art to the instant disclosure.

Respiratory Viral infections are characterized primarily physiologic infection of the respiratory tract. Examples of viruses that infect the respiratory tract are rhinoviruses, influenza viruses (during annual winter epidemics), parainfluenza viruses, respiratory syncytial virus (RSV), enteroviruses, coronaviruses, and certain strains of adenovirus are the main causes of viral respiratory infections.

Coronaviruses and specifically COVID-19 is a new emerging virus affecting humans and is one type of virus amongst a family of viruses that effect man and other species. Coronavirus infection in humans is characterized by a broad array of physiologic and anatomical abnormalities that can result in an acute or chronic condition, for example, including altered glucose disposition, hypertension, retinopathy, abnormal kidney function, abnormal central nervous system function, abnormal cardiac function, abnormal liver function, abnormal platelet activity, abnormal pancreatic function aberrations involving large, medium and small sized vessels, chronic fatigue, rhabdomyolysis, and other co-morbidities and death.

Coronavirus infection and specifically COVID-19 infection has been described initiating in the respiratory tract and involving, sinus, trachea, bronchi and lung function leading to lung injury, hypoxia, shortness of breath, pulmonary embolism. Whether serially or in parallel, blood vessel function, endothelial cell infection, kidney, gastrointestinal, neurological, cardiovascular, pancreatic, injury, skeletal muscle injury and susceptibility to bacterial infection have been described. In addition, associated with coronavirus infection and specifically COVID-19 rhabdomyolysis, and/or hyperactive catabolic syndrome and or acute respiratory distress syndrome and aberrant cytokine expression has been described.

Nucleotide turnover/metabolism—Nucleic acid metabolism is the process by which nucleic acids (DNA and RNA) are synthesized and degraded. Nucleic acids are polymers of nucleotides. Nucleotide synthesis is an anabolic mechanism generally involving the chemical reaction of phosphate, pentose sugar, and a nitrogenous base. Destruction of nucleic acid is a catabolic reaction. Additionally, parts of the nucleotides or nucleobases can be salvaged to recreate new nucleotides. Both synthesis and degradation reactions require enzymes to facilitate the event. Defects or deficiencies in these enzymes can lead to a variety of diseases (Voet 2008)

Purine degradation takes place mainly in the liver of humans and requires an assortment of enzymes to degrade purines to uric acid. First, the nucleotide will lose its phosphate through 5′-nucleotidase. The nucleoside, adenosine, is then deaminated and hydrolyzed to form hypoxanthine via adenosine deaminase and nucleosidase respectively. Hypoxanthine is then oxidized to form xanthine and then uric acid through the action of xanthine oxidase. The other purine nucleoside, guanosine, is cleaved to form guanine. Guanine is then deaminated via guanine deaminase to form xanthine which is then converted to uric acid. Oxygen is the final electron acceptor in the degradation of both purines. Uric acid is then excreted from the body in different forms depending on the animal (Nelson 2008).

Defects in purine catabolism can result in a variety of diseases including gout, which stems from an accumulation of uric acid crystals in various joints.

Inhibitors of the enzyme xanthine oxidase, which converts hypoxanthine to xanthine, and xanthine to uric acid, have been indicated for the treatment of a variety of conditions. For example, the xanthine oxidase inhibitor, allopurinol, is used in the treatment of gout and hyperuricaemia (U.S. Pat. No. 5,484,605). Xanthine oxidase inhibitors have also been proposed for use in suppressing the harmful effects of oxygen radicals that mediate ischaemia-reperfusion injury in a variety of tissues including the heart, lung, kidney, gastrointestinal tract, and brain, and in inflammatory joint diseases such as rheumatoid arthritis. (See for example, U.S. Pat. No. 6,004,966). They have also been reported to be useful in treating excessive resorption of bone. (U.S. Pat. No. 5,674,887).

SARS-CoV-2 (COVID-19) is a lytic virus, which means that during replication in the lung, it can cause destruction of cells within the respiratory tract. Recognition of the virus by innate immune cells can lead to production of pro-inflammatory cytokines and chemokines which have the capacity to cause fever, inflammation, and, in cases of severe disease, vascular and epithelial barrier dysfunction leading to flooding of the alveoli in the lungs (pneumonia), making it hard to breathe and limiting the ability of the lungs to take in oxygen and diffuse carbon dioxide. One early study of 41 patients suggested common symptoms were fever (98%), cough (76%), myalgia or fatigue (44%), and atypical symptoms included sputum (28%), headache (8%), hemoptysis (coughing up blood) (5%), and diarrhea (3%). Nearly half of the patients had difficulty breathing, with most exhibiting this complication about 8 days after first symptoms. Sixty-three percent (63%) of patients showed a reduction in their peripheral blood lymphocyte counts, which could impact the ability of the adaptive immune response to clear the virus. Complications included acute respiratory distress syndrome (29%), acute heart injury (12%), and secondary infections (10%); Thirty-two percent (32%) of the patients required ICU-level care. (The Lancet. 2020; 395: 497-506). An epidemiological study in China demonstrated evidence of asymptomatic infection in some people as well (˜1.2% of patients in this study) (Chinese Journal of Epidemiology. 2020; 41: 145-151)

Lysis of infected tissues such as lung, endothelial cells, blood vessels, heart, cardiovascular system and neurological system, would be anticipated to result in release of cellular contents into the circulation in a scenario similar to Tumor lysis syndrome, and secondarily result in a rise in circulating cell free DNA, then breakdown products of nucleic acid catabolism leading to increased nucleotide, nucleoside, purine and pyrimidine concentrations, hypoxanthine, xanthine and uric acid. It is expected that xanthine oxidase and other enzymes are released into the circulation when cells lyse, including intracellular uric acid and cellular components capable of producing reactive oxygen species (ROS).

The interactions of reactive oxygen species (ROS)—hydrogen peroxide, hydroxy radicals and free oxygen radicals—with biomolecules that result in alterations in cell function or overt cellular damage has been proposed to contribute to pathogenic mechanisms of various disease processes (Freeman 1982, Kinnual 2003, McCord 2002). Xanthine oxidoreductase (XOR) is a Mo-pterin enzyme that serves as the rate-limiting enzyme catalyzing the oxidation of hypoxanthine to xanthine and finally, urate. Upon sulfhydryl oxidation or limited proteolysis, the dehydrogenase (XDH) form of XOR is converted to an oxidase (XO), which utilizes O₂ as the terminal e⁻ acceptor, yielding superoxide (O₂⁻) and hydrogen peroxide (H₂O₂) rather than NADH. Under inflammatory conditions, XO serves as a significant source of O₂⁻ and H₂O₂ in the vasculature (Aslan 2001, Granger 1986, Hare 2003, Leyva 1998, White 1996). During such states, it is has been demonstrated that XDH is released into the circulation, is rapidly (<1 min) converted to XO and binds to positively charged glycosaminoglycans (GAGs) on the surface of vascular endothelial cells (Houston 1999, Parks 1988). In this location, XO can generate ROS that in turn can modulate the bioavailability of nitric oxide (NO) and thus vascular cell signaling (White 1996). Xanthine oxidase displays an affinity for heparin sulfate-containing GAGs on endothelial cells; intravenous administration of heparin mobilizes vascular-associated XO and releases it into the circulation (Houston 1999, Granell 2003).

Coronavirus appears to create a “Viral lysis” syndrome associated with coronavirus has not been previously described, nor has a resulting increase in circulating uric acid levels. It is anticipated that this newly discovered pathway for co-morbidities and mortality associated with cell free DNA and uric acid may affect most body systems. For example, cause Viral Nephropathy, Viral Cardiopathy, Viral Neuropathy or Viral endothelial dysfunction, and Viral Pancreatitis leading to Viral diabetes and the acute and chronic health consequences of such an infection.

Studies by Moreno L et al suggest that monosodium urate crystals exacerbate acute lung injury and the development of pulmonary hypertension, and lung inflammation induced by endotoxin lipopolysaccharide (Moreno 2018).

The novel discoveries identified in the figures of this patent application suggest that hyperuricemia associated with viral infection, coronavirus infection and COVID-19 infection may cause or worsen acute respiratory distress syndrome. Moreover, these finding suggest the need for a uric acid lowering agent.

In addition, in the setting of acute respiratory distress syndrome, when hypoxia, and need for ventilation are needed a uric acid lowering agent or agents may ameliorate the severity and onset of acute respiratory distress syndrome.

Indeed, hyperuricemia may also increase probability of onset or severity of acute respiratory distress syndrome in the setting of sepsis, use of a uric acid lowering agent may be appropriate in this case.

In addition to endothelial infection and renal infection, involvement of the cardiovascular system and neurological system has also been reported.

While respiratory failure associated with COVID19 has received most of the attention, this disease also disrupts the function of the nervous system at many levels. Initially, during the early phases of infection the immune response causes an increase in the level of cytokines that can mediate the headaches associated with the disease (Lancet 2020; 395: 497 and The Journal of Headache and Pain 2020; 21:38). Rare reports of acute necrotizing encephalopathy and encephalitis associated with COVID19 may also be mediated by the so-called “cytokine storm.” (Poyiadji 2020, Ye 2020) Patients with COVID-19 may also experience an increase in blood clots, which increases the risk for stroke caused by blood clots in the brain (Oxley 2020). The longer term effects of COVID19 are uncertain, but the SARS-CoV-2 coronavirus is a member of the betacoronavirus genus which also includes the SARS-CoV-1 and MERS-CoV viruses. A significant concern for the neuroscience community is that like SARS-CoV-1, the SARS-CoV-2 may spread either transsynaptically or by crossing the blood-brain barrier to infect neurons and glia of the central nervous system to produce long-lasting effects following infection of COVID-19 patients (Zubair 2020).

In health, xanthine oxidase is the terminal enzyme in the uric acid pathway and plays a role in the breakdown of nucleic acids converting hypoxanthine into xanthine and finally uric acid. Uric acid excreted primarily by kidneys and secondarily by the gastrointestinal tract.

Cardiovascular tissues are targeted by the COVID-19 coronavirus and represent a key threat to infected individuals. COVID-19 infects the host using the angiotensin converting enzyme 2 (ACE2) receptor, which is expressed in several organs, including lung, heart kidney and intestines. ACE2 receptors are also expressed on endothelial cells (Ferrario 2005).

Moreover, viral infection of individual cells involves key steps including entry of virus into cells, use of viral genome, RNA in the case of coronavirus, to breakdown of intracellular components, then transcription and translation of intracellular components into copies of viral genome, and viral components such as viral capsule and proteins, to generated new viral particles prior to lysis of cells and release of new infectious viral particles. Nucleotides, Nucleosides, Pyrimidine, Purines and other nitrogen sources may be key to ensuring successful viral load, viability and productivity of infection. Xanthine oxidase inhibitor or other inhibitors that decrease the production of uric acid, and nitrogen sources, produced by adenosine catabolism may be particularly useful in treating or preventing coronavirus and specifically COVID-19 infection.

To the knowledge of the inventors, no previous reports of coronavirus leading to hyperuricemia or the association with acute kidney, acute cardiac, acute neurologic, acute pancreatic or acute endothelial injury has occurred. The discovery of early hyperuricemia, and association to these acute conditions and spector of longer-term chronic diseases that may arise from coronavirus infection.

The embodiments that arise from the discoveries described herein support new invention that treating high uric acid levels (above the normal range and/or above 5.5 mg/dL) with uric acid lowering agents may provide protection, against acute and chronic co-morbidities and mortality associated with coronavirus infection.

### Exemplary Embodiments

In particular embodiments, disclosed is a method of treating and preventing COVID-19 infection comprising administering a therapeutically effective amount of an agent capable of reducing uric acid levels in a patient in need of such treatment. A reduction in uric acid would reduce the risk of hypertension, acute kidney, cardiac, liver, vascular, pulmonary, neurological and acute respiratory distress syndrome and morbidity and mortality. Current standards for increased uric acid are 7 mg/dL. However, patients for the above noted 8-10 mg/dL are at increased risk.

In particular embodiments, a method of preventing co-morbidity due to coronavirus infection is disclosed comprising administering a therapeutically effective amount of an agent capable of decreasing uric acid levels in a patient in need of such treatment.

Also provided is a method of treating acute kidney injury due to coronavirus infection comprising administering a therapeutically effective amount of an agent capable of decreasing uric acid levels in a patient in need of such treatment.

In yet another embodiment, disclosed is a method of treating and preventing acute cardiovascular injury due to coronavirus infection comprising administering a therapeutically effective amount of an agent capable of decreasing uric acid levels in a patient in need of such treatment.

Also provided is a method of treating and/or preventing acute cardiac injury due to coronavirus infection comprising administering a therapeutically effective amount of an agent capable of decreasing uric acid levels in a patient in need of such treatment.

In other embodiments, provided is a method of treating and preventing acute lung injury due to coronavirus infection comprising administering a therapeutically effective amount of an agent capable of decreasing uric acid levels in a patient in need of such treatment.

Also disclosed is a method of treating and preventing acute liver injury due to coronavirus infection comprising administering a therapeutically effective amount of an agent capable of decreasing uric acid levels in a patient in need of such treatment.

Also provided is a method of treating and preventing acute pancreas injury due to coronavirus infection comprising administering a therapeutically effective amount of an agent capable of decreasing uric acid levels in a patient in need of such treatment.

Also provided is a method of treating and preventing virally induced metabolic syndrome or diabetes injury due to coronavirus infection comprising administering a therapeutically effective amount of an agent capable of decreasing uric acid levels in a patient in need of such treatment.

An agent capable of reducing uric acid levels by about 0.2 mg/dL. The agent capable of reducing uric acid levels, which is selected from the group consisting of: gene therapy; a xanthine oxidase inhibitor; a uricosuric agent; supplements of the uricase protein and a urate channel inhibitor, or combinations of these agents. Specific examples of agents that are capable of reducing uric acid levels include but are not limited to:
A gene therapy such as one that targets the overexpression of uricase, the enzyme responsible for the breakdown of uric acid to allantoin.
A xanthine oxidase inhibitor, such as allopurinol, carprofen, febuxostat, TMX-049, oxypurinol, NC-2500, 3,4-Dihydroxy-5-nitrobenzaldehyde (DHNB), or other agents.
A uricosuric agent, which is defined as an inhibitor of the organic anion transport channels and/or voltage sensitive transport channels acting in the kidney, such agents include but are not limited to: losartan, benzbromarone, benziodarone, probenecid, sulfinpyrazone, etebenecid, orotic acid, ticrynafen, zoxazolamine, Lesurinad, verinurad, NC-2700.
A supplement of the uricase protein, such as Rasburicase, which might be delivered as a conjugated with polyethylene glycol—pegylated— or another delivery system, such as pegloticase, and that acts in the gastrointestinal tract such as solid oral dosage form of crystalline recombinant oxalate decarboxylase enzyme, such as pegadricase or reloxaliase, or intravenously into the circulation; and
A urate channel inhibitor—is a means for interfering with the uric acid transport mechanism by blocking the influx of uric acid into cells.

Also within the scope of the disclosure is a pharmaceutical composition comprising an agent which stimulates nitric oxide production via endothelial and/or neuronal nitric oxide synthase or a pharmaceutically acceptable salt thereof and the agent capable of reducing uric acid levels or a pharmaceutically acceptable salt thereof as recited above and a pharmaceutical carrier. An agent which stimulates nitric oxide production via endothelial and or neuronal nitric oxide including, but not limited to L-Arginine, L-Citrulline, L-Ornithine, nitrates, and nitrate-mimetics and gene therapy, such as one that targets the over expression of endothelial and/or neuronal nitric oxide synthase.

### Compositions and Formulations

In an aspect, the disclosure provides compositions of xanthine oxidase inhibitors. Compositions of a xanthine oxidase inhibitor of the disclosure may be formulated to ensure maximum activity and bioavailability of the xanthine oxidase inhibitor without increasing any side effects. Purine and non-purine xanthine oxidase inhibitors are free acids, a formulation with an organic base would ensure maximum activity and bioavailability of the xanthine oxidase inhibitor without increasing any side effects.

Particular composition embodiments comprise one or more UALAs including, but not limited to, febuxostat, TMX-049, NC-2500, allopurinol or oxypurinol. The formulations of the composition may have one or more of the following characteristics: physiological compatible pH, stability of formulations with time, on heating, or in humid conditions, a long-lasting conservation, favorable solubility, a better tolerability, enhanced hygroscopicity, desirable physical properties (e.g. compression and flow properties) permitting the manufacture of a formulation useful for pharmaceutical medicinal purposes, a better taste, and formulation to be used in cardiopathic, vascular injury, nephropathic, pancreatic injury, neuropathic and hypertensive patients. Compositions may be formulated where the active xanthine oxidase inhibitor is absorbed more rapidly and to a higher degree resulting in improved bioavailability. Compositions may be formulated to be substantially non-toxic or have lower toxicity. Accordingly, the formulations of xanthine oxidase inhibitors of the disclosure, in particular the formulations of allopurinol and oxypurinol, are expected to be very useful as pharmaceutical composition as compared with previously described parent compounds.

In an aspect, the disclosure provides compositions including at least one uricase enzyme and/or urate oxidase enzyme. Formulations of a uricase of the disclosure are preferably designed to ensure maximum activity and bioavailability of the uricase without increasing any side effects. Uricase in a formulation, with an anti-oxidant or free oxygen radical scavenging molecule would ensure maximum activity and bioavailability of the uricase without increasing any side effects and preferably reduce peroxide and secondarily oxygen radical or secondary other reactive compounds.

Other formulations include uricase, Rasburicase, pegloticase, pegadricase, reloxaliase ,ALLN-346, and may have surprising physiochemical and pharmacological properties. The formulations may have one or more of the following characteristics: physiological compatible pH, stability of formulations with time, on heating, or in humid conditions, a long-lasting conservation, favorable solubility, a better tolerability, enhanced hygroscopicity, desirable physical properties (e.g. compression and flow properties) permitting the manufacture of a formulation useful for pharmaceutical medicinal purposes, a better taste, and formulation to be used in cardiopathic, vascular injury, nephropathic, pancreatic injury, neurologic injury and hypertensive patients. Formulations of the disclosure may provide compositions where the active urate oxidase is absorbed more rapidly and to a higher degree resulting in improved bioavailability. Compositions comprising formulations described herein may be substantially non-toxic or have lower toxicity. Accordingly, the formulations of urate oxidase of the disclosure, in particular the formulations of Rasburicase, pegloticase, pegadricase, reloxaliase, ALLN-346, are expected to be very useful as pharmaceutical agents as compared with previously described parent compounds.

In particular embodiments, the anti-oxidant can also be an organic base such as arginine, choline, L-lysine, D-lysine, glucamine and its N-mono- or N,N-disubstituted derivatives including but not limited to N-methylglucamine, N,N-dimethylglucamine, N-ethylglucamine, N-methyl,N-ethylglucamine, N,N-diethylglucamine, N-β-hydroxyethylglucamine, N-methyl,N-β-hydroxyethylglucamine, and N,N-di-β-hydroxyethylglucamine, benethamine, banzathine, betaine, deanol, diethylamine, 2-(diethylamino)-ethanol, hydrabamine, 4-(2-hydroxyethyl)-morpholine, 1-(2-hydroxyethyl)-pyrrolidine, tromethamine, diethanolamin(2,2″-iminobis(ethanol), ethanolamine (2-aminoethanol), 1H-imidazole, piperazine, triethanolamine (2,2′,2″-nitrilotris(ethanol), N-methylmorpholine, N-ethylmorpholine, pyridine, dialkylanilines, diisopropylcyclohexylamine, tertiary amines (e.g. triethylamine, trimethylamine), diisopropylethylamine, dicyclohexylamine, N-methyl-D-glutamine, 4-pyrrolidinopyridine, dimethylaminopyridine (DMAP), piperidine, isopropylamine, meglumine, N-acetyl-cysteine or caffeine.

In an aspect, the disclosed formulations comprise basic amino acid and/or anti-oxidant formulations comprising Febuxostat, TMX-049, NC-2500, allopurinol and/or oxypurinol.

In a particular aspect, the disclosure provides novel formulations of allopurinol and oxypurinol (in particular, arginine or lysine salts of allopurinol or oxypurinol) which have advantageous properties permitting the manufacture of a stable formulation (e.g. stable over time, on heating, and/or at relative humidity ranges) adapted for medicinal use.

In an embodiment, the disclosure provides formulations of xanthine oxidase inhibitors with glucamine and its N-mono- or N,N-disubstituted derivatives. Examples include but are not limited to N-methylglucamine, N,N-dimethylglucamine, N-ethylglucamine, N-methyl,N-ethylglucamine, N,N-diethylglucamine, N-β-hydroxyethylglucamine, N-methyl,N-β-hydroxyethylglucamine, and N,N-di-β-hydroxyethylglucamine. The formulations may be produced by reacting a glucamine salt with a xanthine oxidase inhibitor.

The present disclosure also relates to a process for preparing the formulations of the disclosure. A process may comprise dissolving a xanthine oxidase inhibitor together with an organic base, optionally with addition of solvent, optionally with an anti-oxidant or optionally with a uricase enzyme. A xanthine oxidase inhibitor may first be dissolved in a solvent and/or a solution of the second, third or fourth substance admixed. It may also be possible to incorporate the xanthine oxidase inhibitor into a solution of the second, third or fourth substance.

The ratio of organic base to xanthine oxidase inhibitor can range from 0.1 to 10.0 molar equivalent organic base to 1.0 molar equivalent of xanthine oxidase inhibitor. In an embodiment the ratio of organic base to xanthine oxidase inhibitor is 5.0:1.0 mole, in particular 3.0:1.0 mole, more particularly 2.0:1.0 mole, and further more particularly 1.0:1.0 mole.

UALA formulations may be in non-crystalline form, micronized form, crystalline or amorphous form, or in a solution or suspension. In another embodiment, the disclosure provides an organic base formulation of a xanthine oxidase inhibitor formed by displacing at least one hydrogen on allopurinol or oxypurinol.

Compositions containing a UALA can be formulated for a desired form of delivery. Formulations include solids (tablets, soft or hard gelatin capsules), semi-solids (gels, creams), or liquids (solutions, colloids, or emulsions). Colloidal carrier systems include microcapsules, emulsions, microspheres, multi-lamellar vesicles, nanocapsules, uni-lamellar vesicles, nanoparticles, microemulsions, and low-density lipoproteins. Formulation systems for parenteral administration include lipid emulsions, liposomes, mixed micellar systems, biodegradable fibers, and fibrin-gels, and biodegradable polymers for implantation. Formulation systems for pulmonary administration include metered dose inhalers, powder inhalers, solutions for inhalation, and liposomes. A composition can be formulated for sustained release (multiple unit disintegrating particles or beads, single unit non-disintegrating system), controlled release (oral osmotic pump), and bioadhesives or liposomes. Controlled release formulations include those, which release intermittently, and those that release continuously. Formulations include liquids for intravenous administration. Formulations may include any combination of liquid or solid formulations administered together or sequentially.

The compositions of the present disclosure typically comprise suitable pharmaceutical carriers, excipients, vehicles, or diluents selected based on the intended form of administration, and consistent with conventional pharmaceutical practices. Suitable pharmaceutical carriers, excipients, vehicles, or diluents are described in the standard text, Remington's Pharmaceutical Sciences (Mack Publishing Company, Easton, Pa., USA 1985). By way of example, for oral administration in the form of a capsule or tablet, the active components can be combined with an oral, non-toxic pharmaceutically acceptable inert carrier such as lactose, starch, sucrose, methyl cellulose, magnesium stearate, glucose, calcium sulfate, dicalcium phosphate, mannitol, sorbitol, and the like. For oral administration in a liquid form, the drug components may be combined with any oral, non-toxic, pharmaceutically acceptable inert carrier such as ethanol, glycerol, water, and the like. Suitable binders (e.g. gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, coloring agents, absorption enhancers, particle coatings (e.g. enteric coatings), lubricants, targeting agents, and any other agents known to one skilled in the art, may also be combined in the compositions or components thereof.

The pharmaceutical compositions disclosed herein can be prepared by per se known methods for the preparation of pharmaceutically acceptable compositions which can be administered to patients, and such that an effective quantity of the active substance is combined in a mixture with a pharmaceutically acceptable carrier, excipient, vehicle, or diluent.

In an embodiment, a composition is formulated so that it remains active at physiologic pH. The composition may be formulated in the pH range 4 to 10, in particular 4 to 7.

### Derivatives

As used herein, solvent refers to any liquid that completely or partially dissolves a solid, liquid, or gaseous solute, resulting in a solution such as but not limited to hexane, benzene, toluene, diethyl ether, chloroform, ethyl acetate, dichloromethane, carbon tetrachloride, 1,4-dioxane, tetrahydrofuran, glyme, diglyme, acetone, acetonitrile, dimethylformamide, dimethyl sulfoxide, dimethylacetamide, or N-methyl-2-pyrrolidone.

It is to be understood that reactants, compounds, solvents, acids, bases, catalysts, agents, reactive groups, or the like may be added individually, simultaneously, separately, and in any order. Furthermore, it is to be understood that reactants, compounds, acids, bases, catalysts, agents, reactive groups, or the like may be pre-dissolved in solution and added as a solution (including, but not limited to, aqueous solutions). In addition, it is to be understood that reactants, compounds, solvents, acids, bases, catalysts, agents, reactive groups, or the like may be in any molar ratio.

It is to be understood that reactants, compounds, solvents, acids, bases, catalysts, agents, reactive groups, or the like may be formed in situ.

### Solvates

The UALAs also includes solvate forms of the agents. The terms used in the claims encompass these forms.

### Polymorphs

The UALAs also include their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

### Prodrugs

Embodiments of the disclosure further include XOI agents and uricosuric agents in prodrug form. Such prodrugs are generally compounds wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

### Applications

The uric acid lowering formulations and compositions disclosed herein may be used to prevent or treat conditions or diseases which require modulation of purine metabolism, serum uric acid concentration or xanthine oxidase, which utilize xanthine oxidase inhibitors to prevent or treat the condition or disease, or which are treatable using a xanthine oxidase inhibitor. Therefore, certain embodiments relate to a method for preventing or treating in a subject a condition or disease which requires modulation of xanthine oxidase or which utilize xanthine oxidase inhibitors to prevent or treat the condition or disease comprising administering a therapeutically effective amount of an organic base, anti-oxidant, and uric acid lowering agent formulation of the disclosure.

Compositions disclosed herein provide a useful means for administering active xanthine oxidase inhibitor compounds to subjects suffering from a condition or disease. A condition or disease includes without limitation a cardiovascular or related disease, ischemia-reperfusion injury in tissues, rheumatoid arthritis, respiratory distress, kidney disease, pancreatic disease, neurological disease, liver disease, sickle cell disease, sepsis, burns, viral infections, hemorrhagic shock, conditions associated with poor cardiac contractility, and conditions associated with excessive resorption of bone associated with viral infection, coronavirus infection or COVID-19 infection. In particular, the condition or disease is hypertension, acute kidney injury, acute cardiac injury, acute neurological injury, acute vascular injury, ischemia reperfusion injury, and diseases that arise from inflammatory, pro-inflammatory, thrombotic and prothrombotic states in which the acute respiratory distress syndrome, hypercatabolic state, cytokine storm, or coagulation cascade is activated.

A formulation of the disclosure or a pharmaceutical composition incorporating such formulation may provide advantageous effects in the treatment of conditions or diseases such as cardiovascular, renal, or neurological, or related diseases, in particular health consequences of COVID-19. The formulations and compositions of the disclosure can be readily adapted to a therapeutic use in the treatment of viral infection and resulting diseases. Thus, contemplated in light of the teachings herein is the use of a formulation or a composition for preventing, and/or ameliorating disease severity, disease symptoms, and/or periodicity of recurrence of a cardiovascular, renal, vascular, neurological, or related disease.

In an embodiment, provided is a composition comprising a basic amino acid, anti-oxidant, uricase formulation of a xanthine oxidase inhibitor that improves acute kidney injury status using MAKE criteria, KIDGO criteria, urine output, serum creatinine concentration, glomerular filtration rate, cardiac efficiency and lowers serum lipid concentrations. Any of these measures concentrations may be decreased or increased by 1-50%, in particular by 1%, 2%, 3%, 4%, 5%, 6%, 7%, 8%, 9%, 10%, 15%, 20%, 25%, 30%, 35%, 40%, or 50%.

The present disclosure contemplates a composition of material which is directed to reducing adverse consequences of free radicals generated in human cells or in the circulatory system relating to viral infection or related disorders by administration of an organic base, anti-oxidant and/or uricase, formulation of a xanthine oxidase inhibitor.

The present disclosure also contemplates a method of treating or protecting kidney function in a mammal in need of enhanced efficiency of renal function and administering to the selected mammal a therapeutically effective amount of an organic base formulation of the disclosure.

The present disclosure further contemplates a method for treatment for a disorder of inflammation in a mammal suffering from or susceptible to the disorder comprising administering to the mammal a therapeutically effective amount of an organic base formulation of the disclosure.

The present disclosure also provides a method for treating viral induced diabetes associated with acute pancreatic injury or a health consequence in a mammal suffering from or susceptible to viral infection, comprising selecting a mammal for treatment of obesity, high blood pressure, metabolic syndrome, diabetes or chronic kidney disease that is suffering from or susceptible to viral or bacterial infection and administering to the selected mammal a therapeutically effective amount of an UALA containing formulation of the disclosure, optionally further comprising an organic base.

In a particular aspect, a method is provided for treating or preventing acute vascular injury in a mammal suffering from or that has suffered vascular injury or endothelial dysfunction comprising administering to the mammal a therapeutically effective amount of an oxypurinol. In another particular aspect, a method is provided for treating pulmonary or acute respiratory distress syndrome (ARDS) in a mammal suffering from or that has suffered vascular injury or endothelial dysfunction comprising administering to the mammal a therapeutically effective amount of an organic base formulation of uricase, or oxypurinol, or anti-oxidant. In a preferred embodiment, the formulation is derived from a basic amino acid, more preferably arginine or lysine.

Another aspect relates to the use of a composition comprising at least one organic base containing formulation of a xanthine oxidase inhibitor of the disclosure for the preparation of a medicament, in particular a medicament for the prevention or treatment of a condition or disease. In an embodiment, the condition or disease is a cardiovascular or related disease. In another aspect, the disclosure relates to the use of effective amounts of at least one organic base containing formulation of a xanthine oxidase inhibitor of the disclosure, in the preparation of a pharmaceutical composition for inhibiting or preventing a condition or disease, in particular a cardiovascular or related disease, in a patient infected with a lytic virus, coronavirus or COVID-19 virus.

According to certain therapeutic methods disclosed herein, a single or combination of more than one of an organic base or an anti-oxidant, or an uricase or a xanthine oxidase inhibitor in a formulation may be administered. Examples of antioxidants include but are not limited to, a flavonoid (such as EGCG, quercetin, catechin and the like), beta-carotene, vitamin C, N-acetyl-cysteine, alpha-lipoic acid, vitamin E, anthocyanin, organic base, and sulforaphane. Thus, a particular therapy can be optimized by selection of an optimal therapeutic combination of formulation of a xanthine oxidase inhibitor, in particular allopurinol or oxypurinol, or optimal cocktail of multiple organic base, uricase, anti-oxidant, anti-inflammatory or xanthine oxidase inhibitors formulations. Optimal compound(s) can be readily selected by those skilled in the art using known in vitro and in vivo assays.

### Administration

The formulations and compositions disclosed herein are useful as therapeutic agents either alone or in conjunction with other therapeutic agents or other forms of treatment. For example, the formulations and compositions may be used in combination with other drugs used to treat cardiovascular diseases including inhibitors of angiotension-converting enzyme (ACE), inotropics, diuretics, and beta blockers. The formulations and compositions of the disclosure may be administered concurrently, separately, or sequentially with other therapeutic agents or therapies. Pharmaceutical compositions may be formulated in a conventional manner using one or more pharmaceutically acceptable carrier, excipient, vehicle, or diluent.

Routes of administration of a therapeutic compound or composition include but are not limited to parenteral (including subcutaneous, intraperitoneal, intrasternal, intravenous, intraarticular injection, infusion, intradermal, and intramuscular); or oral; pulmonary, mucosal (including buccal, sublingual, vaginal, and rectal); topical, transdermal, and the like. Parenteral can be a particularly desirable route of administration.

The methods and uses of the disclosure include both acute and chronic therapies. For example, an formulation or composition of the disclosure can be administered to a patient suffering from chronic metabolic syndrome, hypertension, renal, cardiovascular disease, diabetes or viral pneumonia, bacterial pneumonia, sepsis or cardiogenic shock. A formulation of a xanthine oxidase inhibitor can be administered within about 1, 2, 4, 8, 12, or 24 hours, or more than one day to about 2 to 4 weeks, in particular 2-3 weeks, after a subject has suffered from viral infection induced injury such as acute kidney injury or chronic kidney disease, or diabetic nephropathy, or polycystic kidney disease.

Regular long-term administration of a formulation or composition of the disclosure may be beneficial after a patient has suffered from chronic kidney disease to provide increased cardiovascular health. Therefore, a formulation or composition of the disclosure can be administered on a regular basis to promote enhanced functional capacity, for example, at least, 2, 4, 6, 8, 12, 16, 18, 20, or 24 weeks, or longer such as 6 months, 1 year, 2 years, 3 years, or more after having suffered chronic kidney disease.

In an embodiment, the disclosure teaches a method for treating acute or chronic kidney disease in a subject comprising administering a pharmaceutical composition of the disclosure to the subject, and continuing administration of the formulation until a desirable therapeutic effect is detected in the subject. The desired therapeutic effect may be to improve the efficiency of filtering capacity, glomerular filtration rate, glomerular hypertension, decrease serum creatinine concentration, decrease proteinuria, health, exercise capacity, cardiac output, and/or cardiac efficiency in subjects with viral infection and long-term health consequences of viral infections.

The amounts of formulations of xanthine oxidase inhibitors used in therapeutic methods and compositions of the disclosure will vary according to various factors including but not limited to the specific compounds being utilized, the particular compositions formulated, the mode of application, the site of administration, the age and the body weight of the subject and the condition of the subject to be treated, and ultimately will be decided by the attending physician or veterinarian. Conventional dosing determination tests can be used to ascertain the optimal administration rates for a given protocol of administration. Doses utilized in prior clinical applications for xanthine oxidase inhibitors and/or uricase will provide guidelines for preferred dosing amounts for the methods of the present disclosure.

In an aspect of the disclosure, a composition may contain from about 0.1 to 90% by weight (such as about 0.1 to 20% or about 0.5 to 10%) of the active ingredient.

A xanthine oxidase inhibitor, uricase, anti-oxidant formulation or composition of the disclosure used for prophylactic and therapeutic administration may be sterile. Sterility can be accomplished by filtration through sterile filtration membranes, for example 0.2 micron membranes. Formulations and compositions of the disclosure for prophylactic and therapeutic administration may be stored in unit or multi-dose containers. Dosing may also be arranged in a subject specific manner to provide a predetermined concentration of a xanthine oxidase inhibition activity in the blood. For example, dosing may be adjusted to achieve regular ongoing trough blood levels on the order of from 50 to 1000 ng/ml, in particular, 150 to 55 ng/ml.

An UALA and optional organic base or optional anti-oxidant containing, formulation or composition of the disclosure of a xanthine oxidase inhibitor of the disclosure may be stored in unit or multi-dose containers, for example, sealed ampoules or vials.

The disclosure also provides a pharmaceutical pack or kit comprising one or more containers filled with one or more of the components of a pharmaceutical composition of the disclosure. Associated with a container may be a notice in the form prescribed by a government agency regulating the manufacture, use or sale of pharmaceuticals which notice reflects approval by the agency of manufacture, use or sale for human administration.

Having now described the invention, the same will be more readily understood through reference to the following examples which are provided by way of illustration, and are not intended to be limiting of the present disclosure.

Other enzymes from fungal and viral sources may also increase uric acid by contributing to the adenosine catabolic generation of free oxygen radicals and metabolic products attributed to this pathway including inosine, hypoxanthine, xanthine.

Nucleoside analogue drugs include:
deoxyadenosine analogues: didanosine (ddI)(HIV), vidarabine (antiviral)
adenosine analogues: BCX4430 (Ebola), Remdesivir (Ebola)(Marburg)(Coronavirus)
deoxycytidine analogues: cytarabine (chemotherapy), gemcitabine (Chemotherapy), emtricitabine (FTC)(HIV), lamivudine (3TC)(HIV, hepatitis B), zalcitabine (ddC)(HIV)
guanosine and deoxyguanosine analogues: abacavir (HIV), aciclovir, adefovir, entecavir (hepatitis B)
thymidine and deoxythymidine analogues: stavudine (d4T), telbivudine (hepatitis B), zidovudine (azidothymidine, or AZT)(HIV)
deoxyuridine analogues: idoxuridine, trifluridine
Tenofovir

Related drugs are nucleobase analogs, which don't include a sugar or sugar analog, and nucleotide analogues, which also include phosphate groups.

Uric acid lowering agents can be categorize in to several classes, uricase (for example: Pegloticase or Rasburicase or Pegadricase or reloxaliase or ALLN-346), uricosuric agents (for example, losartan, probenecid, benzbromarone, atorvastatin, fenfibrate, lesinurad, verinurad, sulfinpyrazone, pyrazinamide), or xanthine Oxido-Reductase inhibitors (allopurinol, febuxostat, TMX-049, oxypurinol, NC-2500, NC-2700, NMDA, etc). Indeed, because uric acid and allopurinol but not oxypurinol are potentially building blocks for nucleic acids, the antiviral effect of oxypurinol may require further characterization and prove beneficial in coronavirus infections (Perez-Mazliah 2012).

### Examples

In a VILI animal model, application of high tidal volume mechanical ventilation (HTMV) activated XOR and increased the pulmonary capillary permeability (Abdulnour, 2006). Treatment of endothelial cells directly with ROS or with XO decreases the transendothelial electrical resistance (TEER) and increases the permeability of macromolecules (Shasby, 1985). Oxidative stress is known to induce apoptosis of epithelial cells during VILI (Syrkina, 2008). VILI also induces p38 MAPK mediated inflammatory lung injury (Dolinay, 2008) and activation of p38 increases XOR enzymatic activity. Pharmacological inhibition of p38-XOR attenuates VILI induced lung injury (Le, 2008). These studies indicate a significant role of XOR in ROS mediated lung injury.

### REFERENCES

Abdulnour, R. E. E., Peng, X. Q., Finigan, J. H., Han, E. J., Hasan, E. J., Birukov, K. G., et al. (2006). Mechanical stress activates xanthine oxidoreductase through MAP kinase-dependent pathways. American Journal of Physiology Lung C, 291(3), L345-LL53.CrossRefGoogle Scholar
Shasby, D. M., Lind, S. E., Shasby, S. S., Goldsmith, J. C., & Hunninghake, G. W. (1985). Reversible oxidant-induced increases in albumin transfer across cultured endothelium—alterations in cell-shape and calcium homeostasis. Blood, 65(3), 605-614.PubMedGoogle Scholar
Syrkina, O., Jafari, B., Hales, C. A., & Quinn, D. A. (2008). Oxidant stress mediates inflammation and apoptosis in ventilator-induced lung injury. Respirology, 13(3), 333-340.PubMedCrossRefGoogle Scholar
Dolinay, T., Wu, W., Kaminski, N., Ifedigbo, E., Kaynar, A. M., Szilasi, M., et al. (2008). Mitogen-activated protein kinases regulate susceptibility to ventilator-induced lung injury. PloS One, 3(2), e1601.PubMedPubMedCentralCrossRefGoogle Scholar
Le, A., Damico, R., Damarla, M., Boueiz, A., Pae, H. H., Skirball, J., et al. (2008). Alveolar cell apoptosis is dependent on p38 MAP kinase-mediated activation of xanthine oxidoreductase in ventilator-induced lung injury. Journal of Applied Physiology, 105(4), 1282-1290.
Voet, Donald; Voet, Judith; Pratt, Charlotte (2008). Fundamentals of biochemistry: life at the molecular level (3rd ed.). Hoboken, N.J.:
Nelson, David L.; Cox, Michael M.; Lehninger, Albert L. (2008). Lehninger's Principles of Biochemistry (5 ed.). Macmillan. ISBN 978-0716771081.
Freeman, B. A. and Crapo, J. D. (1982) Lab. Invest. 47, 412-426.
Kinnula, V. L. and Crapo, J. D. (2003) Am.J.Respir.Crit Care Med. 167, 1600-1619.
McCord, J. M. (2002) Methods Enzymol. 349, 331-341.
Aslan, M., Ryan, T. M., Adler, B., Townes, T. M., Parks, D. A., Thompson, J. A., Tousson, A., Gladwin, M. T., Patel, R. P., Tarpey, M. M., Batinic-Haberle, I., White, C. R., and Freeman, B. A. (2001) Proc.Natl.Acad.Sci.U.S.A 98, 15215-15220.
Granger, D. N., Hollwarth, M. E., and Parks, D. A. (1986) Acta Physiol Scand.Suppl 548, 47-63.
Hare, J. M. and Johnson, R. J. (2003) Circulation 107, 1951-1953.
Leyva, F., Anker, S. D., Godsland, I. F., Teixeira, M., Hellewell, P. G., Kox, W. J., Poole-Wilson, P. A., and Coats, A. J. (1998) Eur.Heart J. 19, 1814-1822.
White, C. R., Darley-Usmar, V., Berrington, W. R., McAdams, M., Gore, J. Z., Thompson, J. A., Parks, D. A., Tarpey, M. M., and Freeman, B. A. (1996) Proc.Natl.Acad.Sci.USA 93, 8745-8749.
Houston, M., Estevez, A., Chumley, P., Aslan, M., Marklund, S., Parks, D. A., and Freeman, B. A. (1999) J.Biol. Chem. 274, 4985-4994.
Parks, D. A., Williams, T. K., and Beckman, J. S. (1988) Am.J.Physiol 254, G768-G774.
Granell, S., Gironella, M., Bulbena, O., Panes, J., Mauri, M., Sabater, L., Aparisi, L., Gelpi, E., and Closa, D. (2003) Crit Care Med. 31, 525-530
Moreno, et al, Monsodium urate crystals exacerbate acute lung injury induced by lipopolysaccharide, Eur Resp J 2018
COVID-19—associated Acute Hemorrhagic Necrotizing Encephalopathy: Imaging Features Neo Poyiadji, Gassan Shahin, Daniel Noujaim, Michael Stone, Suresh Patel, and Brent Griffith Radiology 2020 296:2, E119-E120
Mingxiang Ye, Yi Ren, Tangfeng Lv, Encephalitis as a clinical manifestation of COVID-19, Brain, Behavior, and Immunity, Volume 88, 2020, Pages 945-946, ISSN 0889-1591.
Ferrario C M, Jessup J, Chappell M C, et al. Effect of angiotensin-converting enzyme inhibition and angiotensin II receptor blockers on cardiac angiotensin-converting enzyme 2. Circulation 2005; 111: 2605-10.
Zubair A S, McAlpine L S, Gardin T, Farhadian S, Kuruvilla D E, Spudich S. Neuropathogenesis and Neurologic Manifestations of the Coronaviruses in the Age of Coronavirus Disease 2019: A Review. JAMA Neurol. 2020; 77(8):1018-1027. doi: 10.1001/jamaneurol.2020.2065
Oxley T J, Mocco J, Majidi S, Kellner C P, Shoirah H, Singh I P, et al. Large-vessel stroke as a presenting feature of covid-19 in the young. N Engl J Med. 2020;382(20):e60.
Perez-Mazliah D et al, Allopurinol reduced antigen-specific and polyclonal activation of human T-cells, Frontiers in Immunology, Sept 2012.

What is claimed is:

1. A method for treating a health consequence caused by a viral infection in a subject, said method comprising administering a therapeutically effective amount of a uric acid lowering agent (UALA).

2. The method of claim 1, wherein said health consequences comprise an acute kidney injury associated with the viral infection.

3. The method of claim 2, wherein the viral infection is by a coronavirus.

4. The method of claim 1, wherein the health consequence comprises acute renal, vascular, neurological, pancreatic, hepatic, or pulmonary injury caused by a viral infection.

5. The method of any of claims 1-4, further comprising co-administering a basic organic or basic inorganic molecule and wherein said health consequence comprises acute kidney injury.

6. The method of any of claims 1-4, further comprising co-administering a basic organic or basic inorganic molecule, and wherein said health consequence comprises acute vascular injury.

7. The method of any of claims 1-4, further comprising co-administering a basic organic or basic inorganic molecule and wherein said health consequences comprises acute pulmonary injury.

8. The method of claim 1, wherein said administering improves endothelial dysfunction in the subject.

9. The method of any of claims 1-7, further comprising co-administering an anti-inflammatory agent.

10. The method of any of claims 1-7, further comprising co-administering an anti-viral agent.

11. The method of claim 10, wherein the anti-viral agent comprises of didanosine, vidarabine, BCX4430, Remdesivir, emtricitabine, lamivudine, zalcitabine, abacavir, aciclovir, adefovir, entecavir, stavudine, telbivudine, zidovudine, idoxuridine, trifluridine, Tenofovir, or interferons.

12. The method of claim 1, wherein the therapeutically effective amount comprises an amount of an UALA sufficient to a decrease insulin resistance before, after, or during COVID infection.

13. The method as in one of claims 1-10, wherein the administering and/or co-administering comprises intravenous, intramuscular, sublingual, dermal, or oral delivery.

14. The method as in any of claims 1-9, wherein the UALA comprises of a uricase, a xanthine oxidase inhibitor, or a uricosuric agent.

15. The method of claim 16, wherein administering comprises administering a first UALA and co-administering at least one other UALA.

16. A method for treating a health consequence caused by a viral infection in a subject, said method comprising: administering a therapeutically effective amount of a first UALA for a first time period and subsequently administering a therapeutically effective amount of a second UALA for a second time period.

17. The method of claim 16, wherein the first UALA is a uricase.

18. The method of claim 16, wherein the second UALA is a xanthine oxidase inhibitor.

19. The method of claim 14 or 17, wherein the uricase comprises of rasburicase, pegloticase pegdradicase, reloxaliase or ALN-346.

20. A formulation comprising a uricase, a xanthine oxidase inhibitor, and optionally a pharmaceutically acceptable carrier in an amount effective to lower uric acid levels in a subject.

21. The formulation of claim 20, formulated for parenteral delivery.

22. The formulation of claim 20 or 21, formulated for IV delivery.

23. A container into which an amount of the formulation of claim 20 or 21 is disposed.

24. The method of claim 16, where a uricase is formulated with an anti-oxidant or free oxygen radical scavenging molecule.

25. The method of claim 24, wherein the antioxidant comprises a flavonoid (such as EGCG, quercetin, catechin and the like), vitamin C, N-acetyl-cysteine, alpha-lipoic acid, vitamin E, anthocyanin, organic base, and sulforaphane.

26. The formulation of one or more UALA of claim 20 further comprising an organic base that is also an anti-oxidant.

27. The method of any of claims 1-15, further comprising co-administering an antioxidant.
